(19) United States
(12) Patent Application Publication
(10) Pub. No.: US 2014/0088704 A1
(43) Pub. Date: Mar. 27, 2014

(54) FEEDTHROUGH CONSTRUCTIONS AND DEVICES COMPRISING THE SAME

(71) Applicants: Grahame Walling, Newport NSW (AU); Anthony Powell, Bondi Junction NSW (AU)

(72) Inventors: Grahame Walling, Newport NSW (AU); Anthony Powell, Bondi Junction NSW (AU)

(21) Appl. No.: 13/624,686

(22) Filed: Sep. 21, 2012

(51) Int. Cl. A61F 2/18; B23P 11/00; H05K 5/06

(52) U.S. Cl. 623/10; 361/807; 29/428

(57) ABSTRACT

Feedthrough constructions comprise a housing with a feedthrough disposed within a housing opening, wherein the feedthrough is configured to facilitate passage of electrical signals through the housing. The feedthrough has a circumference specially configured to reduce the variation in compressive stress imposed onto the feedthrough by the housing to reduce crack development and/or propagation to thereby extend effective service life.

### FIELD

Feedthrough constructions used for providing one or more inputs/outputs, and devices comprising such feedthrough constructions attached thereto, are disclosed herein.

### BACKGROUND

Devices such as electrical devices comprising a housing and having one or more cables or electrical contacts extending therethrough make use of a feedthrough for the purpose or routing one or more electrical input or output signals to and from the housing. Feedthroughs used in this capacity are configured to fit within an opening through the housing, and comprise one or more passages with electrically-conductive elements therein extending through the feedthrough to facilitate passage of the one or more input or output signals into and/or out of the housing. In certain electrical device applications, the feedthrough forms a hermetic seal with the housing to prevent unwanted elements, such moisture or the like, from entering the housing.

Such known feedthroughs are typically formed from electrically-nonconductive material, and are attached to the surrounding opening in the housing by braze attachment. During installation of such feedthroughs, or during use of the electrical device comprising the same, cracks can develop in the feedthrough itself and/or in the braze material that is interposed between the feedthrough and the housing, and/or between the feedthrough and the braze material, and/or between the housing and the braze material.

The development and existence of such cracks is not desired especially in those applications where a hermetic or leak-tight seal between the feedthrough and the device housing is desired. The presence of such cracks can operate to provide a leak path for unwanted contaminates into the device housing, which can impair proper operation of electrical components or other elements disposed within the housing that can reduce effective service life of the device. Device applications that are especially sensitive to such unwanted impairments in operation include medical devices, such as those that may or may not be implanted.

### SUMMARY

Feedthrough constructions and devices comprising the same as disclosed herein comprise a housing having an opening and a feedthrough disposed within the opening. The housing can comprise one or more electrical components disposed therein, and the housing can be part of a medical device such as one that is implanted into a human body, e.g., such as a component of a hearing prosthesis. In an example, a braze joint is used to attach the feedthrough to the housing opening and provide a hermetic or leak-tight seal therebetween.

In an example, the feedthrough can be formed from an electrically insulative or nonconductive material, and can comprise one or more electrically conductive elements extending therethough, e.g., disposed within passages or the like. The feedthough is configured having a circumference designed to reduce or minimize variation in the compressive stress placed upon it by the housing. In an example, the feedthrough has a substantially convex circumference with an aspect ratio of greater than 1. In an example, the aspect ratio is greater than about 1.2, in the range of between about 1.2 to 10, and preferably between about 2 to 8.

The feedthrough circumference can have a radius of curvature that is different at different locations along the circumference. Additionally, the radius of curvature of opposed tips of the feedthrough circumference can be the same or different, and can be different than the radius of curvature along another section of the circumference.

Feedthroughs and constructions comprising the same as disclosed herein operate to help minimize or eliminate the development and/or propagation of cracks that may occur due to the differences of expansion/contraction characteristics between the housing, braze material, and/or feedthrough, thereby operating to extend the effective service life of constructions comprising the same.

### BRIEF DESCRIPTION OF THE DRAWINGS

These and other features and advantages of improved feedthrough constructions and assemblies comprising the same will be appreciated as the same becomes better understood by reference to the following detailed description when considered in connection with the accompanying drawings.

FIG. 1 is a perspective view of an electrical device comprising a housing and a feedthrough construction disposed therein;

FIG. 2 is plan view of an example feedthrough construction as disclosed herein;

FIGS. 3a and 3b are plan views of example feedthrough constructions as disclosed herein;

FIG. 4 is a perspective view of an example feedthrough construction as disclosed herein;

FIG. 5 is a plan view of an example feedthrough construction of FIG. 4;

FIG. 6 is a plan view of the example feedthrough construction of FIGS. 4 and 5, as disposed within a housing opening and illustrating compressive forces disposed thereon;

FIGS. 7a to 7d are plan views of example feedthrough constructions having different geometric features

FIGS. 8a and 8b are plan views of different example feedthrough constructions having different channel configurations; and

FIG. 9 is a perspective view of a hearing prosthesis comprising an implanted component incorporating the feedthrough construction as disclosed herein.

### DETAILED DESCRIPTION

Improved feedthrough constructions and assemblies comprising the same as disclosed herein are specifically configured in a manner that provides for less variation in the compressive stress that is imposed onto the feedthrough by the housing when installed therein, thereby reducing or eliminating the development and/or propagation of unwanted cracks in a braze material, between the braze material and the housing or feedthrough, and/or in the feedthrough. More specifically, the feedthrough constructions are configured having a substantially convex-shaped circumference with an aspect ratio greater than one. In an example, the feedthrough construction is installed within an opening of device housing, and the device housing imposes a compressive stress onto the feedthrough construction of reduced variation as a result of hoop stress in the housing.

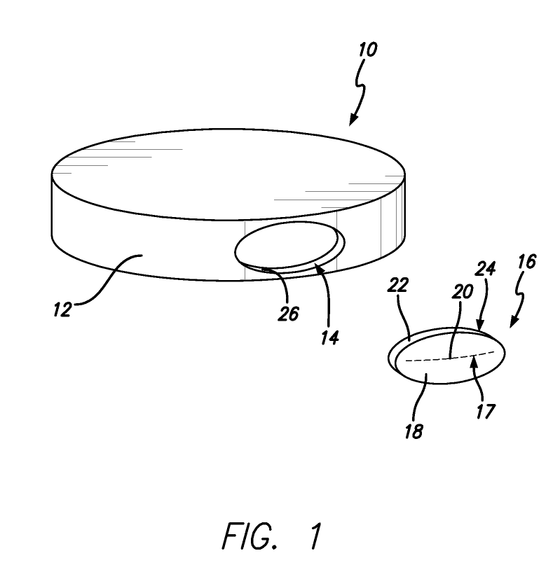

FIG. 1 illustrates a device such as an electrical device 10 comprising a housing 12 that may comprise multiple members. The housing can comprise one or more electrical components (not shown) disposed therein. The housing includes an opening 14 that passes through a portion of the housing which can be a wall structure or the like, and which can be defined by two or more combined members forming the housing. In an example, the housing 12 is configured to accommodate placement of a feedthrough construction or feedthrough 16 therein. While a housing illustrating a single opening and feedthrough has been disclosed and illustrated, it is to be understood that housings as intended to be within the scope of this disclosure may comprise more than one openings and more than one feedthroughs depending on the particular end-use application

The feedthrough construction 16 is configured to include one or more passages 17 extending therethrough from a front surface 18 located outside of the housing to a back surface located within the housing once the feedthrough is installed. The passages can include one or more electrically-conductive elements 20 disposed therein, which can be in the form of wires, cables, vias or the like. In an example, the feedthrough is formed, by molding or machining process, from an electrically-nonconductive or an insulative material such as a ceramic material or the like. A feature of the feedthrough construction is that it facilitates the passage of one or more electrical signals into and out of the housing in a manner that minimizes or eliminates a possible electrical short from occurring between the electrically conductive elements extending through the housing and within the feedthrough construction and the device housing, e.g., when the device housing is formed from a metallic material.

Additionally, in certain device applications calling for a hermetically sealed housing, the feedthrough construction is configured to provide such hermetic or leak-tight seal with both the housing and the electrically-conductive elements extending therethrough. In an example, the feedthrough construction is formed in a manner that provides a leak-tight seal internally therein between it and the electrically-conductive elements. In one example, the electrically-conductive elements are positioned within preformed passages in the feedthrough construction when the feedthrough is a green-state part, e.g., when formed from a ceramic material, and during a heating and/or pressure process the green-state part is sintered forming a compressive seal between it and the electrically-conductive elements disposed in the passages. While a particular feedthrough construction has been disclosed, e.g., having preformed passages, it is to be understood that feedthroughs having non-preformed passages or other types of passages are intended to be within the scope of feedthroughs as disclosed herein. Additionally, the electrically-conductive elements can be provided in many alternative forms capable of passing an electrical signal therethrough, e.g., can be provided in the form of brazed elements and the like, and such is understood to be within the scope of feedthroughs as disclosed herein.

Alternatively, if the feedthrough construction is formed from some other form of electrically-nonconductive material, e.g., a plastic or polymeric material, the leak-tight seal between it and the electrically-conductive elements can be formed during a molding process. Still further, such an internal seal can be provided when the feedthrough construction is in a preformed state by the use of a suitable sealant, adhesive material, or the like.

Referring still to FIG. 1, the feedthrough construction 16 is attached to the housing opening 14 through the use of a suitable sealing material 22 that is interposed between the adjacent outer surface 24 of the feedthrough construction and the inner surface 26 of the housing. In an example, the sealing material is one that is selected to form a strong attachment connection between the feedthrough construction and the housing opening, and can depend on the types of materials used to form each. The housing opening is sized to permit placement of the feedthrough therein and have a small tolerance or space for accommodating placement of the sealing material therebetween to provide a desired level of attachment strength and leak-tight seal is such is desired, e.g., in the case where the housing is to be hermetically sealed.

In an example, e.g., where the feedthrough construction is formed from a ceramic material and the housing is formed from a metallic material, the sealing material can be provided in the form of a metallic braze material. In such an embodiment, a thin layer of braze material is provided between the feedthrough construction and housing opening and is heated to its melting temperature to flow therebetween and seal any gaps between the housing and feedthrough. Upon cooling, the braze material forms a leak-tight braze joint that operates to form a strong/leak-tight attachment between the housing and the feedthrough construction.

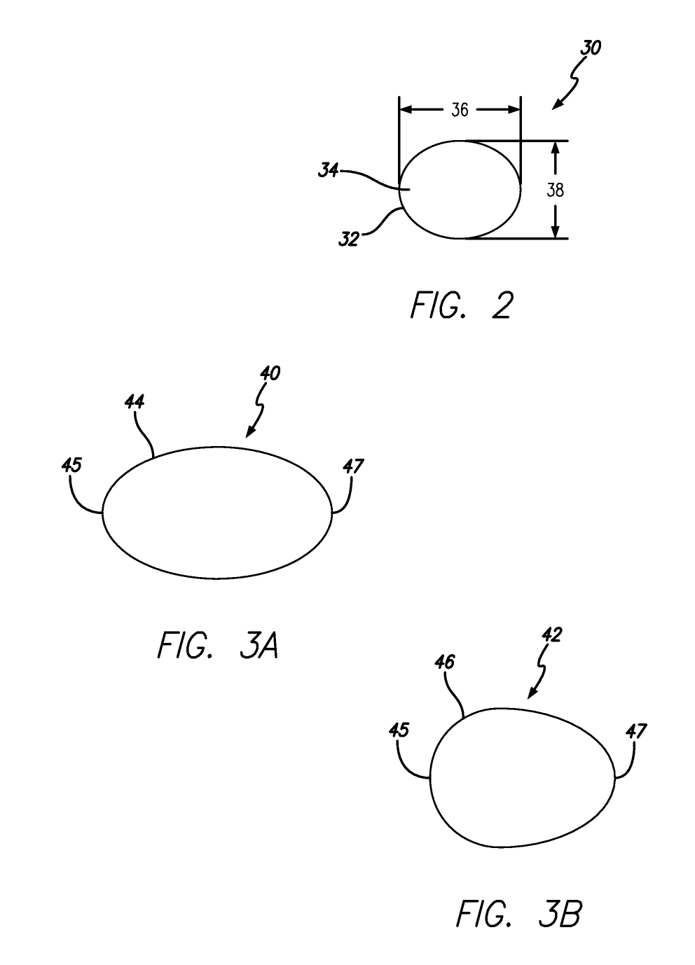

FIG. 2 illustrates in plan view an example feedthrough construction 30 as disclosed herein, showing the general configuration of its circumference 32 that defines the thickness of the feedthrough between its top surface 34 and back surface (not shown). A feature of feedthrough constructions as disclosed herein is that they have a substantially convex-shaped circumference that is defined by an aspect ratio greater than one. As used herein, the term “aspect ratio” is defined as the maximum dimension 36 of the circumference 32 divided by the minimum dimension 38 of the circumference. In an example, the aspect ratio of such feedthrough constructions are typically greater than about 1.2, and can be in the range between about 1.2 to 10, and preferably in the range of from about 2 to 8.

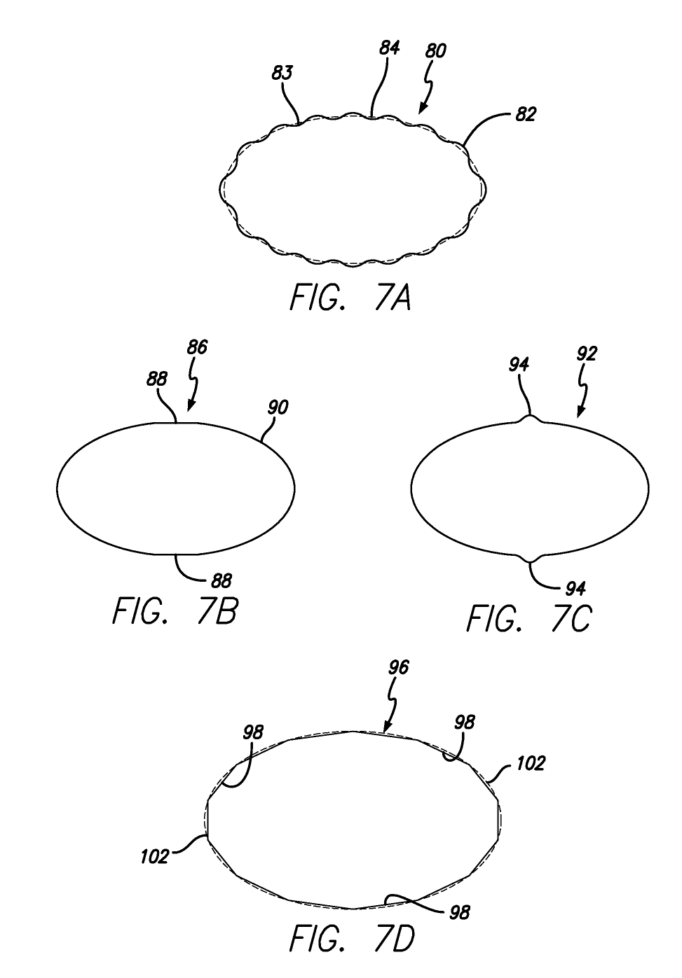

A further feature of feedthrough constructions as disclosed herein is that they are configured to have a substantially convex-shaped circumference. As used herein the term “substantially” is understood to mean that the while the overall configuration of the circumference is one largely viewed as having a convex curvature, the circumference may include one or more sections or segments that are not convex but, because of the minority and/or minor nature of such non-convex sections or segments they do not detract from the overall substantially convex configuration. Examples of feedthrough constructions illustrating this point are provided in FIGS. 7a to 7d that are described below.

In an example, the convex curvature of the feedthrough is maintained along substantially all points along the circumference, but the curvature is not constant around the entire circumference. An example configuration meeting this qualification is one where the feedthrough construction is configured having an elliptical circumference or profile. Thus, while the curvature can be constant in some sections of the circumference, the curvature does not have to be constant or the same for all of the sections.

A feedthrough construction having a substantially convex circumference operates to cause the compressive stresses or forced imposed on the feedthrough and/or braze joint by the housing opening to have less or reduced variation therealong, which is not the case with feedthrough constructions having long straight sections, or non-radiused corners or tips along its profile. This is beneficial feature as such residual compressive stresses help to create and maintain a hermetic or leak-tight seal. Additionally, the ability to reduce or lessen variations in compressive stresses along the circumference of the feedthrough operates to help prevent and/or reduce crack propagation through the feedthrough construction or braze joint or feedthrough seal, thereby improving the robustness of the assembly comprising the housing and the feedthrough construction to a mechanical stress, for example due to an impact event or the like.

FIGS. 3a and 3b illustrate examples of feedthrough constructions 40 and 42 respectively comprising differently-shaped circumferences or profiles 44 and 46 that each meets both criteria of having an aspect ratio greater than one, and that have a substantially convex circumference. Specifically, FIG. 3a illustrates a feedthrough construction 40 having an elliptically-shaped circumference 44, where the radius of curvature for each of the tips 45 and 47 is the same, and wherein the circumference is symmetrically-shaped about an axis running perpendicular to a longitudinal axis through the construction. FIG. 3b illustrates a feedthrough construction 42 having an egg-shaped circumference 46, where the radius of curvature for one tip 45 is different from that of an opposite tip 47, and wherein the circumference is nonsymmetrically-shaped or asymmetrical about an axis running perpendicular to a longitudinal axis through the construction. For both of these feedthrough constructions the circumference is convex everywhere.

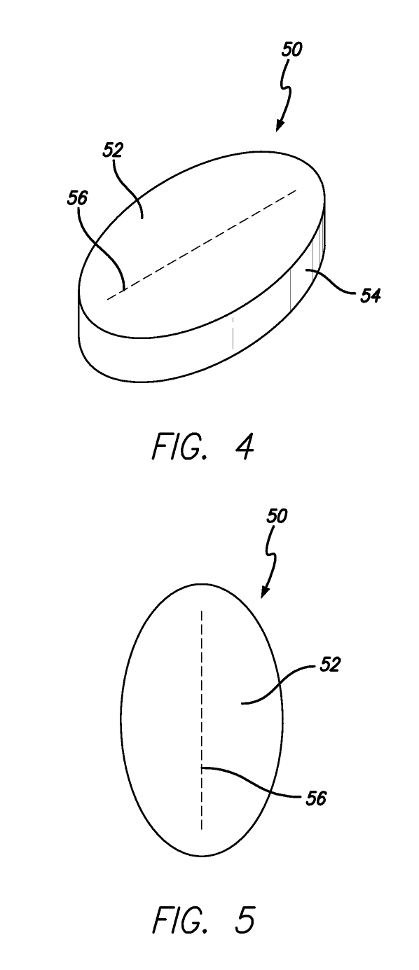

FIGS. 4 and 5 illustrate example feedthrough constructions 50 comprising a generally planar front surface 52 and back surface (not shown), and having a thickness 54 extending therebetween. The thickness of the feedthrough construction can and will vary depending on the particular device and the housing opening thickness. Generally, the feedthrough construction thickness is designed to provide a desired attachment and leak-tight seal within the opening of a particular housing.

The feedthrough constructions 50 comprise a number of passages 56 extending between the front and back surfaces through the thickness and containing electrically-conductive elements disposed therein. In such example, the passages and electrically-conductive elements extend are positioned sequentially along longitudinal dimension of the construction. While a particular arrangement of passages have been disclosed and illustrated for this example for purposes of reference, it is to be understood that the number of passages and the arrangement of the same in the feedthrough construction can and will vary depending on the particular use application, and all such variations are within the scope of feedthrough constructions as disclosed herein.

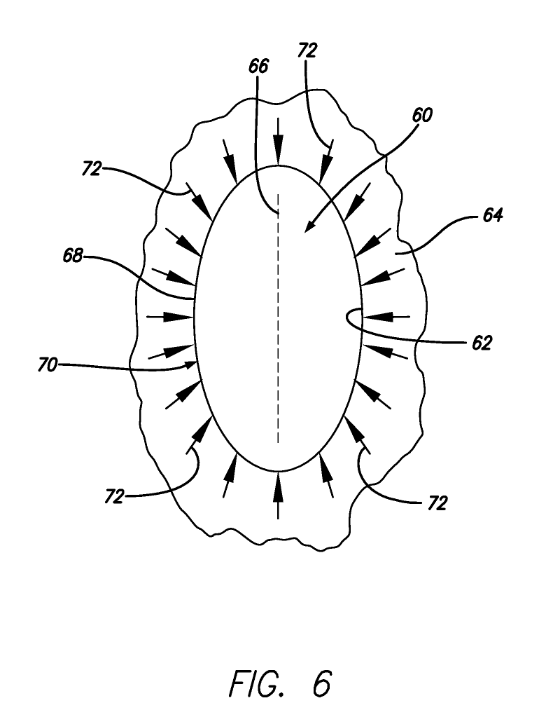

FIG. 6 illustrates an example feedthrough construction 60 as disposed within an opening 62 of a housing 64, and comprising the passages 66 with the electrically-conductive elements extending therethrough. The feedthrough construction is attached to the housing by a braze joint 68 interposed between the feedthrough construction circumference 70 and the housing opening 62. The braze joint also operates to provide a hermetic or leak-tight seal between the feedthrough construction and housing opening. Arrows 72 are positioned around the feedthrough construction and illustrate a compressive stress or force of reduced variation that is distributed onto and along the full circumference of the feedthrough construction by the housing.

Such reduced variation of the compressive stress is a result of the particular shape of the feedthrough construction circumference as disclosed herein when the housing contracts onto the feedthrough construction when cooling from a brazing process because of the different coefficients of thermal expansion between the housing and the feedthrough construction, e.g., when the housing is formed from a metallic material and the feedthrough construction is formed from a ceramic material. Such reduced variation in compressive stress is desired because it helps to achieve a hermetic seal between the housing opening and the feedthrough constructions, and also helps to maintain such a seal by reducing or preventing crack propagation through the feedthrough construction and/or the braze joint.

FIGS. 7a to 7d illustrate different embodiments of feedthrough constructions each having an aspect ratio of greater than one, and having a substantially convex circumference. In particular, these examples are provided to illustrate how feedthroughs as disclosed herein can include deviations along the circumference that are not convex, while still having an overall circumference profile that meets the qualification of having a substantially convex profile.

FIG. 7a illustrates a feedthrough construction 80 having a combination of convex surface features 82 and concave surface features 83 along the circumference shown by a solid line 82, while the overall circumference of the feedthrough has a is substantially convex profile as shown by a dotted line 84. FIG. 7b illustrates a feedthrough construction 86 comprising flat sections 88 along the opposed ends of the circumference 90 that does not detract from the overall profile of the circumference being substantially convex. FIG. 7c illustrates a feedthrough construction 92 comprising protrusions 94, which may be used to register or guide orientation and/or alignment of the feedthrough construction with the housing opening, that does not detract from the overall profile of the circumference being substantially convex. FIG. 7d illustrates a feedthrough construction 96 comprising a series of straight segments 98 shown by a solid line 100, while the overall profile of the circumference is substantially convex as shown by a dotted line 102. These are but a few examples provided for purposes of reference of feedthroughs having non-convex elements yet having a circumference with an overall profile that is substantially convex, and it is to be understood that other variations of the same exist and are intended to be within the scope of feedthrough constructions as disclosed herein.

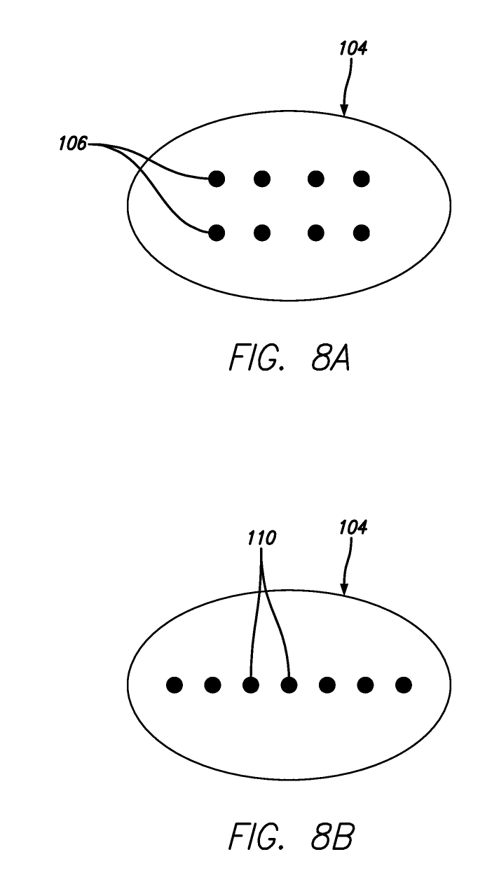

FIGS. 8a and 8b illustrate embodiments of feedthrough constructions showing examples of different passage arrangements in multi-channel feedthroughs. FIG. 8a illustrates a feedthrough construction 104 comprising passages 106 that are arranged in two parallel series. This particular arrangement illustrates passages configured to accommodate a 2×4 channel feedthrough embodiment. FIG. 8b illustrates a feedthrough construction 108 comprising passages 110 that are arranged in a single series. This particular arrangement illustrates passages configured to accommodate a 1×7 channel feedthrough embodiment. It is to be understood that these examples of passage arrangement have been provided for reference, and that many different passages arrangements can exist to accommodate the particular feedthrough construction use application, and all such variations are within the scope of feedthrough constructions as disclosed herein.

Feedthrough constructions as disclosed herein can be used with a variety of different electronic devices designed for different end-use applications. In an example, such feedthrough constructions can be used in conjunction with medical devices that can be worn by a user or implanted into the body of the user. Examples of such medical devices include hearing prosthesis, cardio rhythm or pacing devices, muscular tissue stimulation devices, in neurological stimulation devices, and the like.

In an example, such medical device can be a component of a hearing prosthesis, such as an implanted component of a cochlear implant or the like.

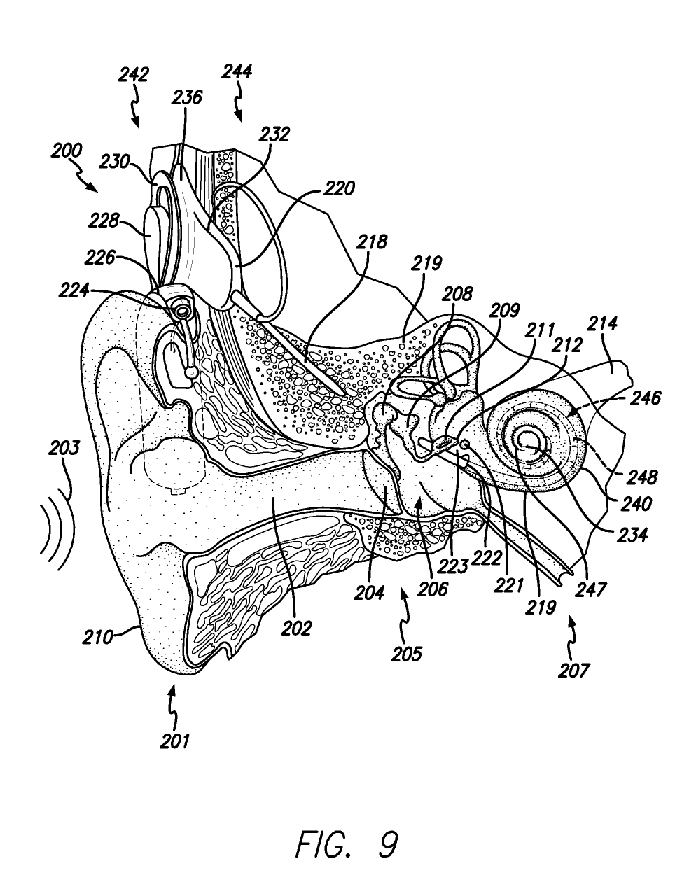

FIG. 9 illustrates a cochlear implant system 200 includes an internal component 244 typically having an internal receiver/transceiver unit 232, a stimulator unit 220, and an elongate stimulating assembly 218 comprising the electrode construction as disclosed herein. The internal receiver/transceiver unit 232 permits the cochlear implant system 200 to receive and/or transmit signals to an external device 226 and includes an internal coil 236, and preferably, a magnet (not shown) fixed relative to the internal coil 236. Internal receiver unit 232 and stimulator unit 220 are hermetically sealed within a biocompatible housing, sometimes collectively referred to as a stimulator/receiver unit. The magnets facilitate the operational alignment of the external and internal coils, enabling internal coil 236 to receive power and stimulation data from external coil 130. The feedthrough construction as disclosed herein is disposed within an opening through the stimulator unit 220 and is hermetically sealed thereto.

Elongate stimulating assembly 218 has a proximal end connected to stimulator unit 220, and a distal end implanted in cochlea 240. Stimulating assembly 218 extends from stimulator unit 220 to cochlea 240 through mastoid bone 219. In certain examples, external coil 230 transmits electrical signals (e.g., power and stimulation data) to internal coil 236 via a radio frequency (RF) link, as noted above. Internal coil 236 is typically a wire antenna coil comprised of multiple turns of electrically insulated single-strand or multi-strand platinum or gold wire. The electrical insulation of internal coil 236 is provided by a flexible silicone molding (not shown). In use, implantable receiver unit 232 may be positioned in a recess of the temporal bone adjacent auricle 210 of the recipient. Various types of energy transfer, such as infrared (IR), electromagnetic, capacitive and inductive transfer, may be used to transfer the power and/or data from external device to cochlear implant.

While a particular hearing prosthesis implant has been described and illustrated, it is to be understood that feedthroughs and constructions comprising the same as disclosed herein can be embodied in hearing prosthesis implants other than that specifically described.

In an example, where the feedthrough construction is used in conjunction with such an implanted medical device, the housing is formed from a metallic material such as titanium and the like, and the feedthrough construction is formed from a ceramic material. In such example, the braze material can be formed from metals or metal alloys conventionally used for providing a desired attachment between the housing and the feedthrough, and can vary depending on the particular materials used for the housing and feedthrough. In an example, e.g., where the housing is formed from titanium and the feedthrough is formed from a ceramic material, the braze material is a TiCuNi alloy. In certain applications that braze material may be formed from gold or an alloy thereof. The housing opening is configured to compliment the shape of the feedthrough construction circumference and is sized to provide a tolerance of about 50 microns therebetween. In such particular embodiment, the feedthrough construction is sized having a longitudinal dimension of between about 6 to 12 mm, having a width of up to about 2 mm, and having a thickness of up to about 2 mm. A ring of the braze material is provided around an edge of the feedthrough circumference, and is melted to fill the gap and provide the desired hermetic seal.

Certain examples of feedthrough constructions, devices, and/or assemblies comprising the same have been disclosed. While such constructions, devices, and/or assemblies have been described with respect to a limited number of examples, the specific features of one example construction should not be attributed to other examples of the construction. No single example is representative of all aspects of constructions and devices comprising the same as disclosed herein. In some examples, the construction devices comprising the same comprise features or content different from and/or not mentioned herein, for example, the feedthrough construction is attached to the housing opening by way of a shrink fit instead of brazing. Variations and modifications from the described examples exist. Finally, any number disclosed herein should be construed to mean approximate, regardless of whether the word “about” or “approximately” is used in describing the number. The appended claims intend to cover all those modifications and variations as falling within the scope of the feedthrough constructions and devices comprising the same as disclosed herein.

What is claimed is:

1. A device comprising:
a housing comprising one or more electrical components disposed therein; and
a feedthrough disposed within an opening through the housing, the feedthrough being formed from an electrically insulating material and comprising one or more passages having an electrically conductive element disposed therein, wherein the feedthrough has a substantially convex circumference with an aspect ratio of greater than 1.

2. The device as recited in claim 1 wherein a leak-tight seal exists between the feedthrough and the opening.

3. The device as recited in claim 1 wherein the aspect ratio is greater than about 1.2.

4. The device as recited in claim 1 wherein the feedthrough circumference has a radius of curvature that is different at different locations along the circumference.

5. The device as recited in claim 4 wherein the radius of curvature along opposed tips of the circumference is different than the radius of curvature along another section of the circumference.

6. The device as recited in claim 5 wherein the radius of curvature along the tips of the circumference is less than the radius of curvature along another section of the circumference.

7. The device as recited in claim 1 wherein the aspect ratio is between about 1.2 and 10.

8. The device as recite in claim 1 wherein the aspect ratio is between about 2 and 10.

9. The device as recited in claim 1 wherein the feedthrough is attached to the housing opening by a braze joint.

10. The device as recited in claim 1 wherein feedthrough comprises more than one passages, and wherein the passages are arranged in a series along a longitudinal dimension of the feedthrough.

11. The device as recited in claim 1 wherein the housing is hermetically sealed and is implantable within a human body.

12. The device as recited in claim 1 wherein the device is a medical device.

13. The device as recited in claim 12 wherein the medical device is a hearing prosthesis.

14. The device as recited in claim 13 wherein the housing is an hermetically-sealed implantable component of the hearing prosthesis.

15. The device as recited in claim 1 wherein the feedthrough circumference comprises one or more surface features that are not convex.

16. A device comprising:
a housing comprising one or more electrical components disposed therein, wherein the housing is hermetically sealed and is implantable within a human body; and
a feedthrough disposed within an opening through the housing and forming a leak-tight seal therewith, the feedthrough being formed from an electrically insulating material and comprising one or more electrically conductive elements extending therethrough, wherein the feedthrough has a substantially convex circumference with an aspect ratio that is greater than about 1.2.

17. The device as recited in claim 16 wherein the aspect ratio is between about 1.2 and 10.

18. The device as recited in claim 16 wherein the aspect ratio is between about 2 and 8.

19. The device as recited in claim 16 comprising a braze joint attaching the feedthrough to the housing opening.

20. The device as recited in claim 16 wherein the feedthrough circumference has opposed tips that have the same radius of curvature.

21. The device as recited in claim 16 wherein the feedthrough circumference comprises one or more non-convex segments.

22. The devices as recited in claim 16 wherein the device is a hearing prosthesis.

23. The device as recited in claim 22 wherein the housing is an implantable component of the hearing prosthesis.

24. The device as recited in claim 23 wherein implanted component is part of a cochlear implant.

25. An implantable hearing prosthesis comprising:
an external component that is positionable outside of a human body and comprising an external coil; and
an internal component that is implantable into a human body and comprising a coil, and a stimulating assembly having a hermetically-sealed housing, the housing including an opening disposed therethrough and a feedthrough disposed within the opening, wherein the feedthrough comprises one or more electrically conductive elements extending therethrough, the feedthrough having a substantially convex circumference that has an aspect ratio greater than one.

26. The hearing prosthesis as recited in claim 25 wherein the feedthrough has an aspect ratio of greater than about 1.2.

27. The hearing prosthesis as recited in claim 25 wherein the feedthrough has an aspect ratio of between about 1.2 and 10.

28. The hearing prosthesis as recited in claim 25 wherein the feedthrough has an aspect ratio of between about 2 and 8.

29. The hearing prosthesis as recited in claim 25 comprising a braze joint connected to the feedthrough and housing.

30. The hearing prosthesis as recited in claim 25 wherein the feedthrough circumference is symmetrical taken through a section perpendicular to a longitudinal axis.

31. The hearing prosthesis as recited in claim 25 wherein the feedthrough circumference is asymmetrical taken through a section perpendicular to a longitudinal axis.

32. The hearing prosthesis as recited in claim 25 wherein one or more sections of the feedthrough circumference have a non-convex shape.

33. A method for making a construction comprising a feedthrough and a device housing comprising the steps of:
constructing the feedthrough having a circumference with an aspect ratio greater than one, the feedthrough having one or more electrically conductive elements extending therethrough; and
attaching the feedthrough to an opening of the device housing;
wherein the circumference is substantially convex.

34. The method as recited in claim 33 wherein during the step of constructing, the feedthrough is shaped to have an aspect ratio of greater than about 1.2.

35. The method as recited in claim 33 wherein during the step of constructing, the feedthrough is shaped to have an aspect ratio of between about 1.2 to 10.

36. The method as recited in claim 33 wherein during the step of attaching, the feedthrough is brazed with the housing to provide a hermetic seal therebetween.

37. The method as recited in claim 33 wherein the housing is part of a hearing prosthesis.

38. The method as recited in claim 33 wherein the housing is implantable within a recipient's body.

39. The method as recited in claim 33 wherein during the step of constructing, the feedthrough circumference is shaped having one or more non-convex surface features.

40. The method as recited in claim 33 wherein during the step of constructing, the feedthrough circumference is shaped having opposed tips with the same radius of curvature.

41. The method as recited in claim 33 wherein during the step of constructing, the feedthrough circumference is shaped having a symmetric configuration taken along a section running perpendicular to a longitudinal axis.
